(19) United States
(12) Patent Application Publication
(10) Pub. No.: US 2008/0058770 A1
(43) Pub. Date: Mar. 6, 2008

(54) DEVICE FOR THE EXTRACTION OF LIQUIDS

(75) Inventors: Jochen HEINZ, Flintbek (DE); Florian WILDENHAHN, Kiel (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: TRANSCOJECT GMBH & CO. KG, Neumunster (DE)

(21) Appl. No.: 11/847,386

(22) Filed: Aug. 30, 2007

(30) Foreign Application Priority Data
Aug. 30, 2006 (DE) 10 2006 040 670.2

(51) Int. Cl. A61M 39/04

(52) U.S. Cl. 604/533; 604/256

(57) ABSTRACT

An extraction device for the extraction of a liquid from a medical receptacle is equipped with a connector, which is designed for connection with the medical receptacle and for creation of a conduit connection into the medical receptacle. Furthermore, a component is provided on the extraction device, which is movably mounted to the connector. This component has a plurality of connections for a liquid outlet, wherein the connector and the component are movable relative to each other, such that they create a conduit connection between the medical receptacle and optionally one of the connections.

### BACKGROUND OF THE INVENTION

The invention concerns a device for the extraction of a liquid from a medical receptacle.

For the extraction of liquids from medical receptacles or vessels, such as infusion bottles, receptacles for contrast mediums and the like, it is known to penetrate the rubber membrane closures of these receptacles with injection syringes and, as a consequence, to deliver the contents of the receptacle to the patient through a flexible hose line. Furthermore, a connection adapter with a concave arbor to penetrate the membrane and with a Luer-lock connector to connect a flexible hose line is known. In this case, on the other end of the flexible hose there is another Luer-lock connector provided for the connection of an injection syringe. The disadvantage of these connection adapters is that they are only convenient for one use, because otherwise it cannot be excluded that germs and other impurities can reach the receptacles by passing via the flexible hose and, above all, via the connection of the flexible hose. For this reason, for repeated extraction from bottles, they are penetrated by several different adaptors one after the other. This causes the disadvantage that rubber particles are formed and carried off and that the rubber membrane becomes permeable because of the several penetrations.

### BRIEF SUMMARY OF THE INVENTION

Against this background the present invention has an object of creating a device for the extraction of a liquid from a medical receptacle which is designed for multiple use, wherein an impurity of the receptacle, mainly caused by germs or damage to the closure of the receptacle caused by wear of the rubber, can be reliably excluded, and the extraction device can be produced and handled in a simple way.

The device for the extraction of a liquid from a medical receptacle, according to this invention, is equipped with a connector constructed for connection with the receptacle and for creation of a conduit connection with the receptacle. Besides, the extraction device is equipped with a component movably mounted with the connector and having a plurality of connections for the outlet of the liquid. According to this invention, the connector and the component are movable relative to each the other, so that a conduit connection between the receptacle and—optionally—at least with one of the connections can be created.

The extraction device is typically designed for the connection with such medical receptacles, in which on a membrane placed in an extraction-position on the underside (in receptacles like this it is normally fixed by an aluminum collar) an opening for the extraction is created by perforation. Through this opening the liquid contained in the receptacle can flow out due to the gravitational force. According to this, the extraction device is assembled on the undersurface of the medical receptacle. For weight reasons, the device is expediently made of synthetic (plastic) material or of similar light but dimensionally stable materials.

The fastening of the extraction device on the receptacle is carried out by the connector, which can be constructed to create a form-fit and/or force-fit connection with the receptacle. A special form of the connector allows for the fastening of the extraction device with the receptacle by gripping around the aluminum collar designed for the fixing of the membrane.

Furthermore, there is a component—specifically a concave arbor—being part of the connector and designed for the penetration of the membrane on the receptacle, wherein this arbor creates the conduit connection between the receptacle and the connector. In the assembled condition of the extraction device, it extends into the extraction device.

According to this invention, the outlet of the liquid from the extraction device is not intended to be on the connector, but is carried out optionally through one or more connections of the component movable relative to the connector. Here, the connector and the component are movable relative to each other, so that a conduit connection is created between the inner side of the receptacle and at least one connection, that is, one liquid outlet of the device is formed. Furthermore, the extraction device allows for a relative movement of the component and the connector toward such a position in which there is no liquid-conducting connection between the inner side of the receptacle and the liquid outlet of the extraction device. The consequence is that, apart from the component, the conduit connection inside the outlet device can be closed as well.

According to the number of connections forming a liquid outlet on the component, several liquid-conducting connections can be created by this extraction device between the receptacle and a liquid outlet. In this way, it is advantageous if the extraction device is used several times without having to use a liquid outlet or a connector of the extraction device for more than one time. Thus, for example, the connector can be coupled with a medical receptacle during the first use of this device. After that, by an appropriate positioning of the component to the connector, a conduit connection from the inside of the receptacle to one of the connections of the component is created. As a consequence, the liquid in the receptacle can be carried to the patient through a flexible tube joined to the connector. After the conclusion of the liquid extraction, the component and the connector can be moved relative to each other, so that the component closes the conduit connection inside the extraction device. By this means, it is not necessary to uncouple the extraction device from the receptacle in case of non-use. If the extraction device is to be used for a second time, the connector and the component have to be moved relative to each other, so that a conduit connection is created from the inside of the receptacle to one of the connectors of the component which have not yet been used. This procedure can be repeated several times, according to the number of connections the component is equipped with.

It is advantageous if the component is introduced and hold in the connector in a form-fit manner. Thus, the connector is designed for retaining the component, wherein it is beneficial if a part of the connector engages behind the component in a form-fit way. Further, the connector forms a guide for the component in which the component can be moved to create a conduit connection between the receptacle and the liquid outlet. In this context, the guide indicates the direction of the relative movement of the connector and the component.

The connections with the component are purposefully arranged in a row in the direction of the relative movement of the connector and the component. This arrangement guarantees that with multiple uses of the extraction device, by a relative movement between the connector and the component, conduit connections are created in a successive order between the inside of the receptacle and the liquid outlet. In this context, the liquid outlet of the corresponding conduit connections is formed by successive connections, which are consecutively arranged on the component in the movement direction. Preferably, on the connector and the component there are markings showing the relative positions of the connector and the component. In these positions there are liquid-guiding connections between the inside of the receptacle and the connection arranged on the component. Another advantage is that the connector and the component can form a snap-on connection in the corresponding positions creating a conduit connection with a connection. In this context, for example, a projection formed on the connector can snap into a recess complementary to the projection or into the corresponding recess on the connector.

In a preferred embodiment of the extraction device of this invention, the component has a circular form. In this context, the liquid outlets and the connections are respectively arranged and distributed over the circumference of the annulus. In this extraction device the component having the shape of a revolver and placed on the connector is arranged around its central axis and can be moved by allowing a simple turning movement of the liquid outlets, that is, a connection of the component before the liquid conduit arranged in the connector. This also allows the simple creation of a conduit connection between the receptacle and the liquid outlet. Furthermore, this embodiment of the component also allows a space-saving form of the extraction device.

In order to avoid, on one hand, penetration of germs and other impurities into the medical receptacle through the connector and the connections arranged on the component and, on the other hand, leakage in the area of the connection of the component with the connector, a seal is preferably arranged between the connector and the component, in order to seal off the connector from the component and protect it from the penetration of liquids and germs. This seal is beneficially arranged in the area of the extraction device where the component is guided and held in the connector in a form-fit manner.

Particularly when the component is formed as a circular ring, wherein the liquid outlets and the connectors are arranged and distributed over the circumference of the annulus, a seal for this can comprise a rubber ring having on the front surface a series of elevations having the shape of burls. These form a flow channel and mesh with the liquid outlets in a form-fit manner. In this context, the circular distribution of the elevations having the shape of burls and their number correspond to the circular distribution and the number of the connections arranged on the component.

In order to be able to penetrate a membrane of a medical receptacle by the extraction device and to create a conduit connection between the receptacle and the extraction device, the connector is equipped with a concave arbor arranged on the side not exposed to the component but on the side exposed to the receptacle.

An advantage offered by the extraction device according to this invention is its equipment with at least two connectors preferentially arranged in an interlocked position and differing primarily in their shape and/or dimension. Thus, the connectors can be constructed as part of all coupling systems used in medical technology in order to connect conduits, wherein the extraction device allows, for example, the connection of flexible hose lines with different connectors.

Another advantage offered by an embodiment of this invention concerning the extraction device comprises the connections of the component producing the liquid outlets respectively being arranged as Luer-lock connections. In this manner, flexible hose lines or syringes equipped with Luer-lock connections can easily and rapidly be attached to the connections.

Preferably, on every liquid outlet there is a germ-impenetrable and beneficially one-way closure. These closures are expediently constructed in order to close in a germ-impenetrable way at least the outlet openings of the connections of the component. In this manner, the contamination of the extraction device and as a consequence of the medical receptacle by germs and impurities can be avoided. Another advantage of the use of one-way closures is that connections having already been used are indicated by the lack of closures, in order to avoid the multiple use of a connection.

The closures are preferably formed by a seal sheet covering the outlet openings of the connections in a germ-impenetrable manner. A further preferred embodiment comprises closures formed by locking caps.

In order to avoid the multiple use of a liquid outlet, the extraction device according to this invention preferably allows only one relative movement of the connector and the component in one movement direction. For example, the connector and the component can be equipped with wedge-shaped detente elements, formed so that they guarantee the movement of the connector and the component relative to each other in a fixed direction only, by blocking movement in the opposite direction. In case of a component with a circular arrangement of the liquid outlets in an endless row, the use of blocks arranged on the connector and on the component can also avoid the liquid outlets being used more than one time for the extraction of a liquid.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is an exploded perspective view of one embodiment of an extraction device according to the invention:

FIG. 2 is a perspective view of the extraction device according to FIG. 1 in the assembled condition; and

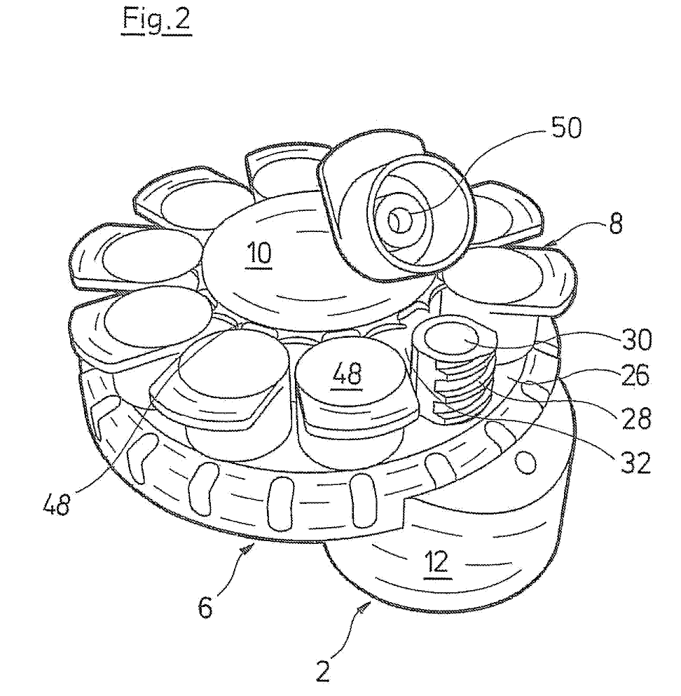

FIG. 3 is a partially cut away, perspective view of the assembled extraction device according to FIG. 2.

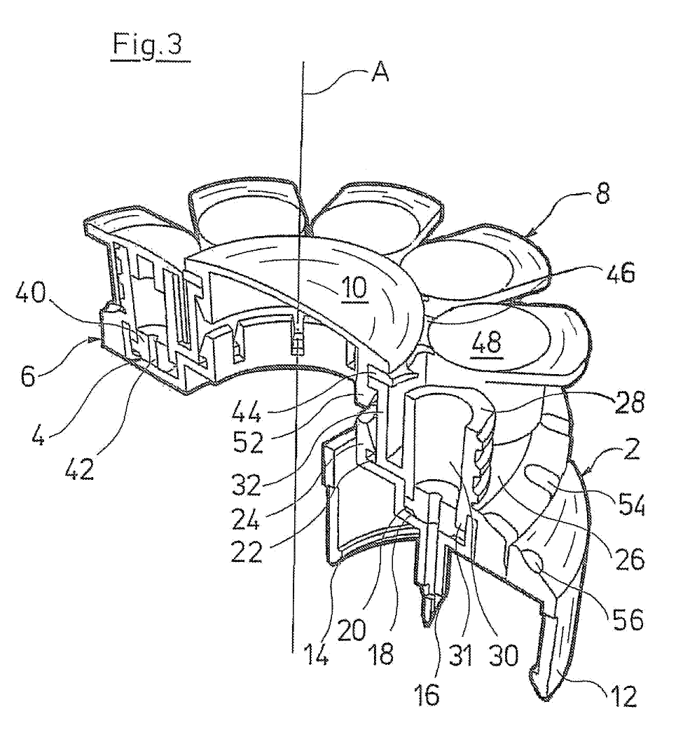

### DETAILED DESCRIPTION OF THE INVENTION

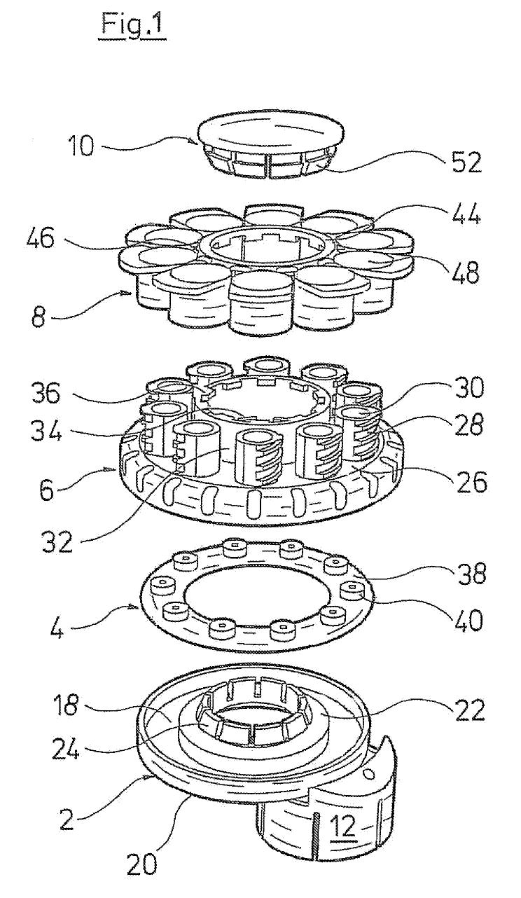

The figures and in particular the exploded view in FIG. 1 show that the extraction device according to this invention comprises essentially a connector 2, a seal 4, a component 6, a locking cap part 8 and a fastening cap 10.

The connector 2, made as a plastic, injection molded part, forms the sleeve-shaped connection cap 12 to connect the extraction device with one of the medical receptacles, which are not represented in the figures, for example an infusion bottle or a contrast medium receptacle.

In this context the connection cap 12 is designed for connection with receptacles having an extraction area similar to a narrowed bottle neck, on an extraction opening is closed by a membrane, which is fastened on the receptacle by a crimp. This fastening is carried out by an aluminum collar gripping around the outer wall of the receptacle in the extraction area.

The connection cap 12, having an inner contour corresponding essentially to the outer contour of the extraction area, is attached on this extraction area of the receptacle. Here, a projection 14, arranged in the region of the open end of the connection cap 12 on its inner wall and extends over the entire inner circumference of the connection cap 12 in the direction of its central axis), engages the aluminum collar of the medical receptacle and thus allows a form-fit fastening of the connection cap 12 and consequently the extraction device to the receptacle.

In order to guide the projection 14 past the aluminum collar of the receptacle, the wall of the connection cap 12 is slit several times in the direction of its longitudinal extension, distributed over the circumference, which in connection with a certain elasticity of the plastic material of the connection cap 12 leads to a flexibility of the wall in the radial direction, whereby the inner cross section of the connection cap 12 can be extended in the area of the open end. After having passed the aluminum collar, it can return to its original cross-sectional dimension.

In order to penetrate the membrane of the medical receptacle and to create a conduit connection there is a concave arbor 16 arranged concentrically to the connection cap 12. Its free and pointed end is oriented toward the open end of the connection cap 12. The other end of the concave arbor 16 empties into a planar base area 18 of a guide 20, in which the component 6 is introduced into the extraction device in an assembled condition.

Apart from the connection cap 12, the guide 20 also forms a part of the connector 2. It is arranged on the front side of this connection cap 12 facing away from the open end of the connection cap 12. The guide 20 is arranged eccentrically to the connection cap 12, so that the connection cap 12 and the guide 20 connected thereto only overlap in a partial segment.

The base area 18 of the guide 20 is constructed in a circular form and is limited by lateral walls on both its inner and outer circumference. These lateral walls extend vertically to the base area 18 in the direction facing away from the open end of the connection cap 12. Starting from the inner lateral wall of the base area 18, there is another circular surface 22 parallel to the base area 18 arranged in the direction of the central axis A of the guide 20. Starting from the inner circumference of the surface 22 there are detente elements 24 arranged on the entire circumference oriented vertically to the surface 22 in a direction facing away from the open end of the connection cap 12.

The guide 20 is designed for retaining and supporting the component 6, which is constructed as a plastic injection-molded part, like the connector 2. The component 6 also has a circular form and corresponds to the guide 20, wherein an outer wall of the component 6 extends in the direction of its central axis A and then has a turning point, so that it forms a connection area 26 extending radially in the direction of the central axis A of the component.

On the connection area 26, which is parallel to the base area 18, there are ten connections 28 distributed over its circumference, arranged with their longitudinal axes parallel to the central axis A of the component 6. These connections 28 are respectively equipped with a flow channel 30 parallel to the central axis A of the component 6. The flow channels 30 are led through the wall of the component 6 forming the connection area 26 and lengthened by tube-shaped connecting pieces 31 on the front side of the wall facing away from the connection area 26. The connections 28 form the liquid outlets of the extraction device and are each formed as Luer-lock connection, so that the connections 28 can be connected with a corresponding Luer-lock connection, for example with a syringe or a tube hose line.

Starting from the inner edge of the connection area 26, facing the central axis A of the component 6, there is a wall 32 extending vertically to this connection area 26 in the direction of the longitudinal extension of the connections 28. In the area of the inner circumference of this wall 32 there are detente elements 34, respectively distributed over the entire circumference in the area of the corner formed by the connection area 26 and the wall 32 and also in the area of the free ends of the wall 32 there are detente elements 36, wherein both the detente elements 34 and the detente elements 36 project in the direction of the central axis of the component 6.

In the assembled condition of the extraction device, the seal 4 is disposed between the guide 20 of the connector 2 and the component 6. The seal 4 is a sealing-ring made of rubber with a planar and circular base body 38, whose radial dimensions essentially correspond to the dimensions of the base area 18 of the guide 20. On one of the faces of the base body 38 there are ten elevations 40 having the shape of burls extending vertically to the face. The elevations 40 in general have a cylindrical shape. The diameter of the elevations 40 is complementary to the inner diameters of the flow channels 30 in the connections 28 of the component 6. Every elevation 40 is broken through by one of the flow channels 42 running in their longitudinal direction.

In the case of the connector 2 assembled with the seal 4 and the component 6, the elevations 40 of the seal 4 engage in the tube-shaped connecting piece 31 of the component 6. The planar and laminar face of the base body 38 of the seal 4 lies then on the base area 18 of the guide 20. The inner side of the outer wall of the component 6 lies against the outer side of the outer wall of the guide 20, limiting the base area 18. In this way, this wall limiting the base area 18 on the outer side engages behind the connecting piece 31 of the component 6, which is diametrically opposed to it. With its outer side it also comes to lie against the inner wall limiting the base area 18 of the guide 20. In the area between the connections 28 and the wall 32, the wall of the component 6 which forms the connection area 26, bears on the surface 22 of the guide 20 with its opposite side. The form-fit fastening of the component 6 to the guide 20 and to the connector 2 occurs by engaging the detente elements 24 of the guide 20 behind the detente elements 34 of the component 6. This form-fit closure guarantees the cohesion between connector 2 and component 6 and also allows a movement of the component 6 with the seal 4 arranged thereon inside the guide 20 of the connector 2.

The locking cap part 8 comprises an inner ring 44 on whose outer circumference there are ten locking caps 48 fixed respectively by a thin bar 46. The bars 46 form set break points, by which the individual locking caps 48 can be detached from the ring 44.

In the assembled condition the locking cap part 8 is arranged such that the locking caps 48 overlap the connections 28 of the component 6. In this way, one of the cylindrical pins 50 arranged in the interior spaces of the locking caps 48, the outer dimensions of the pin corresponding with the inner dimensions of a flow channel 30 in the connector 28, engages with a flow channel 30. In this way, the liquid outlets of the connections 28 are closed in a germ-impenetrable manner. In its assembled condition, the ring 44 of the locking cap part 8 bears on the free end of the wall 32 of the component 6.

The fastening of the locking cap part 8 on the component 6 is carried out with a fastening cap 10. The fastening cap 10 forms a circular top cover, wherein detente elements 52 are arranged on one front side of the top cover next to the outer circumference and extending normal the front side. The fastening cap 10 is pushed toward the opening formed by the wall 32 of the component 6, so that the detente elements 52 of the fastening cap 10 engage behind the detente elements 36 arranged on the inner side of the wall 32. In this way, the ring 44 of the locking cap part 8 lying on the free end of the wall 32 becomes fixed between the wall 32 of the component 6 and the top cover of the fastening cap 10.

In order to extract a liquid from a medical receptacle, the connection cap 12 of the connector 2 must be placed over the extraction area of the medical receptacle, so that the concave arbor 16 penetrates the membrane of the medical receptacle and the projection 14 of the connection cap 12 engages behind the aluminum collar to fasten the membrane on the receptacle, and so that the extraction device is fastened on the receptacle.

Before the extraction from the receptacle, the component 6 and the seal 4 connected thereto are turned in the guide 20 of the connector 2, such that the flow path of the concave arbor 16 is closed by the base body 38 of the seal 4. In this way, no liquid can escape the receptacle.

In this condition one of the locking caps 48 is pulled and broken off from one of the connections 28 of the component 6. On the dissembled Luer-lock of this connector 28, it is now possible to fasten a flexible tube with a corresponding Luer-lock connection.

After that the component 6 and the seal 4 are turned toward the connector 2, so that the flow path through the concave arbor 16, the flow channel 42 of the seal 4 and the flow channel 30 formed in the corresponding connector 28 form a fluid conduit from the medical receptacle to the connected flexible tube. Through this tube the liquid can be conducted to the patient. The turning position required here for the component 6 relative to the connector 2 is shown by the markings 54 on the component 6, which are assigned to each connector 28, whereby the corresponding marking 54 and the marking 56 must be brought to the cover in order to create a conduit connection in the radial direction of the connector 2.

In order to conclude the extraction of the liquid from the receptacle the component 6 is turned relative to the connector 2, so that the marking 56 and the marking 54 are set in a radial alignment to each other. In this position the seal 4 closes the flow path by the concave arbor 16, so that no liquid can escape the receptacle and no germs or impurities can enter the receptacle from outside through the component 6. The connector 28 of the component 6 currently in use can be recognized by the lack of the locking cap 48. In order to use the extraction device again, another locking cap 48 is detached from the connector 38 and the above-described procedure is repeated.

Not represented in the figures, there can be provided in a contact area between the connector 2 and the component 6 wedge-shaped detente elements, whose formation facilitates the movement of the component 6 inside the guide 20 only in one direction, that is in the clockwise or counter-clockwise direction.

In connection with a strike pairing, consisting of one strike arranged on the component 6 and, for example, a second strike correspondingly arranged on the guide 20, a turning movement of the component 6 inside the guide 20 exceeding an angle of 360° can be excluded, whereby a multiple use of the connections 28 is prevented.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A device for extraction of a liquid from a medical receptacle, the device comprising a connector (2) for connection with the medical receptacle and to create a conduit connection into the medical receptacle, and a component (6) movably bearing thereon, wherein the component (6) has a plurality of connections (28) for outlet of the liquid, and wherein the connector (2) and the component (6) are movable relative to each other, such that they create a conduit connection between the medical receptacle and optionally at least to one of the connections (28).

2. The extraction device according to claim 1, wherein the component (6) is guided and held inside the component (6) in a form-fit manner.

3. The extraction device according to claim 1, wherein the connections (28) on the component (6) are arranged in a row in a direction of the relative movement of the connector (2) and the component (6).

4. The extraction device according to claim 1, wherein the component (6) has a circular ring shape, and wherein the connections (28) are distributed and arranged around the circumference of the circular ring.

5. The extraction device according to claim 1, wherein a seal (4) is provided between the connector (2) and the component (6) seals off the connector (2) against liquids and germs relative to the component (6).

6. The extraction device according to claim 5, wherein the seal (4) comprises a rubber ring, and wherein the rubber ring has burl-shaped elevations (40) on one surface, the elevations each forming a flow channel (42) and engaging with the connections (28) in a form-fit manner.

7. The extraction device according to claim 1, wherein the connector (2) has a concave arbor (16) on a side facing away from the component (6).

8. The extraction device according to claim 1, wherein at least two connections are provided, which are preferentially lockable and have a different form and/or dimension.

9. The extraction device according to claim 1, wherein the connections (28) are formed as Luer-lock connections.

10. The extraction device according to claim 1, wherein each connection (28) is preferentially equipped with a lock against germs which cannot be used again after its displacement.

11. The extraction device according to claim 10, wherein the locks comprises a seal sheet.

12. The extraction device according to claim 10, wherein the locks comprises locking caps (48).
